(19) United States
(12) Patent Application Publication
(10) Pub. No.: US 2021/0361412 A1
(43) Pub. Date: Nov. 25, 2021

(54) INTRAOCULAR LENS INJECTOR WITH SPEED REGULATION

(71) Applicant: Carl Zeiss Meditec AG, Jena (DE)

(72) Inventor: Jaime Zacharias, Santiago de Chile (CL)

(21) Appl. No.: 17/380,399

(22) Filed: Jul. 20, 2021

Related U.S. Application Data
(63) Continuation of application No. PCT/EP2020/000023, filed on Jan. 22, 2020.
(60) Provisional application No. 62/795,531, filed on Jan. 22, 2019.
(60) Provisional application No. 62/799,654, filed on Jan. 31, 2019.

(51) Int. Cl. A61F 2/16; A61F 2/48

(52) U.S. Cl. CPC A61F 2/167; A61F 2/48

(57) ABSTRACT

An intraocular lens (IOL) injector with speed regulation for inserting an IOL into a capsular bag of an eye includes an injector body, a cavity, a cartridge ending at a distal end in an injection nozzle, the injector body, the cavity and the cartridge having a channel extending in axial direction, and an axially movable pusher rod advanced by a spring through a shaft, the pusher rod being arranged in the channel of the injector body for pushing the IOL in axial direction out of the cavity and into the cartridge the injection nozzle and finally into the capsular bag of the eye. The pusher rod is engaged with a mechanical speed regulator to limit the maximum speed of injection of the IOL into the eye up to a preset maximum speed for IOL injection, and the speed regulator system is responsive to a user actuation on a lever.

### CROSS REFERENCE TO RELATED APPLICATIONS

This application claims priority to U.S. Provisional Patent Application Nos. 62/795,531 filed on Jan. 22, 2019, 62/799,654 filed on Jan. 31, 2019, and PCT Application No. PCT/EP2020/000023, filed on Jan. 22, 2020, and the entire contents of these applications are incorporated herein by reference.

### TECHNICAL FIELD

The disclosure relates to an intraocular lens injector for inserting an intraocular lens (IOL) into a capsular bag of a human eye, and in particular to an intraocular lens injector with a speed regulator system that is based on a mechanical timer mechanism. The disclosure further relates to a method for controllably releasing an IOL with an intraocular lens injector.

### BACKGROUND

IOLs are medical devices implanted inside the eye. With recent advances in IOL technology, cataract surgery has transitioned from being solely a treatment for visual rehabilitation to also being a refractive procedure with the aim of improving visual function and ultimately the patient's quality of life. IOLs that increase visual function are for example aspheric IOLs and toric IOLs. Aspheric IOLs compensate a spherical aberration of the cornea. Toric IOLs correct corneal astigmatism. IOLs also correct distance vision. The performance of new IOL designs highly depends on the position of the IOL in the optical system of the eye. Depending on the type of correction, even little decentration and tilt of an IOL in the eye may decrease visual quality.

Foldable IOLs made from silicone, soft acrylics, and hydrogels have become increasingly popular because of the ability to fold or roll such soft lenses and to insert them through a small incision. Such IOLs typically consist of a small plastic lens with plastic side struts, called haptics, to hold the lens in place within the capsular bag inside the eye. Such an IOL may be, e.g., a single piece (or one-piece) IOL. The haptic material of a single piece IOL is the same as the optical material. There are also three-piece IOLs with haptics made of a material, e.g., polymethylmethacrylate (PMMA) filaments, which is harder than the optical material.

During surgery, the lens of the patient's eye is removed from the capsular bag of the eye and replaced by the IOL. A most common technique for inserting a foldable IOL into the capsular bag of the eye is through an injector. Such injectors use a plunger to “squeeze” the IOL through a cartridge into the eye. Single piece acrylic and silicone plate haptic IOLs are the simplest to use with injectors. These designs have haptics that are sturdy and to a certain extent are resistant to damage from the plunger, as it forcefully pushes the IOL through the cartridge. In addition, common injectors are not reliable and easy to handle.

Two completely different injector systems are known for inserting a soft and foldable lens. One injector system consists of an injector body (a handpiece) and a separate cartridge, whereby the injector body is reusable or disposable, and the separate cartridge is disposable. Another injector system consists of a preloaded injector, which means the lens is stored within the preloaded injector. Typically, the surgeon pushes a plunger with one finger to inject the IOL into the eye while using the other fingers to stabilize and orient the nozzle into a surgical incision. This simultaneous holding and injecting maneuver can reduce control of the speed of the injection, with a probability of a too rapid and uncontrolled insertion leading to complications such as iris rupture, capsule rupture, and vitreous loss just to mention some.

### SUMMARY

It is an objective of the present disclosure to provide an IOL injector, a method for controllably releasing an IOL with an IOL injector, and in particular an IOL injector that can be handled in an easy, safe, and controllable manner.

It is also an objective of the present disclosure to limit the speed of releasing the IOL to a predetermined maximum speed to avoid fast and uncontrolled IOL injections, to increase accuracy of IOL delivery, and prevent complications.

It is further an objective of the present disclosure to provide an advantageous and controlled method to release an IOL from the IOL injector into the eye.

The objective is achieved by providing an IOL injector for releasing an IOL to be injected into a capsular bag of an eye, the IOL injector having a proximal end and a distal end, the IOL injector including an injector body defining a longitudinal axis and including a hollow shaft, a spring arranged in the hollow shaft, a cartridge arranged on the injector body and defining a cavity for holding the IOL, an injection nozzle arranged on the cartridge at the distal end of the IOL injector, a channel extending through the injector body, the cavity, the cartridge, and the injection nozzle in an axial direction along the longitudinal axis, a mechanical speed regulator system configured to control a speed of the releasing of the IOL, and a pusher rod engaged via the hollow shaft with the mechanical speed regulator system and arranged in the channel movably in the axial direction by a force generated by the spring to advance the IOL in the axial direction towards the distal end of the IOL injector out of the cavity through the cartridge and the injection nozzle.

According to an aspect of the disclosure, the spring is a coil spring. The mechanical speed regulator system is configured to limit the speed of the pusher rod for releasing the IOL to a preset maximum speed.

According to another aspect of the disclosure, the IOL injector further includes a control lever arranged on the injector body in communication with the mechanical speed regulator system, and the mechanical speed regulator system is responsive to an actuation of the control lever. The mechanical speed regulator system includes a first compound gear, a second compound gear, and a third compound gear, the first compound gear includes a first pinion and the hollow shaft includes a rack, the first pinion and the rack form a rack and pinion mechanism to mechanically couple the mechanical speed regulator system with the hollow shaft. The first, second, and third compound gears form a gear train, and the gear train has a speed multiplying configuration.

According to yet another aspect of the disclosure, the spring is a pre-tensed spiral spring arranged on the first compound gear of the mechanical speed regulator system.

According to an aspect of the disclosure, the mechanical speed regulator system further includes an anchor mechanism with an anchor, and the third compound gear includes an escapement wheel mechanically engaging with the anchor. The anchor mechanism includes an anchor shaft defining a center of oscillation of the anchor, the anchor has an anchor body, and the anchor body is mounted on the anchor shaft and swivels about the anchor shaft.

According to another aspect of the disclosure, the anchor mechanism further includes a pendulum arm arranged on the anchor body and a mass arranged on an end of the pendulum arm facing away from the anchor body.

According to another aspect of the disclosure, the third compound gear of the mechanical speed regulator system includes a flywheel, the flywheel includes at least one centrifugal speed limiter to generate an inertial component to permit to incrementally control the speed of the pusher rod when the pusher rod is moved in the axial direction, and the speed of the pusher rod is incrementally controlled by depressing the control lever.

The flywheel has a flywheel body, the at least one centrifugal speed limiter includes a radially flexible arm mounted on the flywheel body and a centrifugal mass arranged on an end of the radially flexible arm facing away from the flywheel body.

According to a further aspect of the disclosure, the flywheel includes air resistive fins configured to limit a speed of rotation of the flywheel through air friction to a maximum speed of rotation.

According to yet another aspect of the disclosure, the IOL injector of claim 10, further includes a friction brake including a friction band in contact with a friction wheel built into the second compound gear, a friction band anchor stud, and a gear lock. One end of the friction band is fixed by the friction band anchor stud and another end of the friction band is anchored to the gear lock such that an incremental rotation of the gear lock progressively loosens the friction band from the friction wheel thereby progressively releasing the friction brake and progressively changing a speed of rotation of the flywheel.

According to a further aspect of the disclosure, the mechanical speed regulator system includes an escapement rack arranged on the hollow shaft, an anchor operatively coupled with the escapement rack, a control rod, and an anchor shaft, the anchor including an anchor body rotatably mounted on the anchor shaft, a pendulum arm mounted on the anchor body, and a pendulum mass having a mass value and being arranged on the pendulum arm. The pendulum arm is configured to act as the spring and has a pendulum arm length and an arm spring constant. The pendulum arm, the pendulum mass, and the anchor body form a pendulum and the pendulum arm length, the arm spring constant, and the mass value are preset such that the pendulum swings at a rate at which the hollow shaft and the pusher rod advance at a maximum speed.

According to an aspect of the disclosure, the maximum speed is a speed at 3 millimeters per second (mm/s).

According to yet another aspect of the disclosure, the injector body includes a speed regulator housing, and the mechanical speed regulator system is arranged in the speed regulator housing.

The object of the disclosure is further achieved by a method for controllably releasing an IOL with an IOL injector, the IOL injector having a proximal end and a distal end and including an injector body defining a longitudinal axis and including a hollow shaft, a spring arranged in the hollow shaft, a cartridge arranged on the injector body and defining a cavity for holding an IOL, an injection nozzle arranged on the cartridge at the distal end of the IOL injector, a channel extending through the injector body, the cavity, the cartridge, and the injection nozzle in an axial direction along the longitudinal axis, a mechanical speed regulator system, and a pusher rod engaged via the hollow shaft with the mechanical speed regulator system and arranged in the channel movably in the axial direction by a force generated by the spring, and a control lever, the method including the steps of advancing the IOL by the pusher rod in the axial direction towards the distal end of the IOL injector out of the cavity through the cartridge and the injection nozzle thereby releasing the IOL, and controlling a speed of the releasing of the IOL.

According to another aspect of the disclosure, the method further includes limiting the speed of the pusher rod for releasing the IOL to a preset maximum speed. The preset maximum speed is the speed at 3 millimeters per second (mm/s).

According to a further aspect of the disclosure, the method further includes incrementally controlling the speed of the pusher rod by depressing the control lever.

Further, in some exemplary embodiments, the speed of injection below the preset maximum speed of injection can be proportionally controllable by the action of a user depressing a control lever.

In an exemplary embodiment, the IOL injector includes a pusher rod that is powered by stored energy from a spring and released by an IOL release lever. One advantage of the IOL injector according to the disclosure is therefore that a surgeon is very familiar with handling such an injector, having a release lever to push for both, initiating the IOL delivery into the eye and also, to regulate the speed of injection up to a maximum speed limit built into the speed limiting mechanism of the injector. In addition, the IOL injector according to an aspect of the disclosure can consist of an injector body (a hand piece) and a separate cartridge, whereby the injector body is reusable or disposable and the separate cartridge is disposable.

The IOL injector according to yet another the disclosure can also be configured as a preloaded injector. The handling of both systems is very similar, so that a surgeon may use both systems, a preloaded system or a loadable system, without additional training. This also increases flexibility and safety, and also decreases the cost for stocking and handling the injector system.

### BRIEF DESCRIPTION OF THE DRAWINGS

The disclosure will now be described with reference to the drawings wherein:

FIG. 1 is a perspective view of an IOL injector according to a first exemplary embodiment of the disclosure;

FIG. 2 is a side view of the IOL injector shown in FIG. 1;

FIG. 3 is a top view of the IOL injector shown in FIG. 1;

FIG. 4 is a bottom view of the IOL injector shown in FIG. 1;

FIG. 5 is a cross-sectional side view of the IOL injector shown in FIG. 1;

FIG. 6 is a cross sectional side view of the side of the IOL injector opposite to the side shown in FIG. 5;

FIG. 7 is a side view of the IOL injector shown in FIG. 1 with the housing removed;

FIG. 8 is a perspective side view of the IOL injector shown in FIG. 1 with the housing removed;

FIG. 9 is a perspective top view of an IOL injector according to a second exemplary embodiment of the disclosure;

FIG. 10 is a perspective view of the IOL injector shown in FIG. 9 with a partially removed housing;

FIG. 11 is a perspective view of the IOL injector shown in FIG. 9 with the housing removed;

FIG. 12 is a perspective view of the IOL injector shown in FIG. 9 with a safety tab in a lock position;

FIG. 13 is a perspective view of the IOL injector shown in FIG. 9 with a safety tab in an operate position;

FIG. 14 is a side view of the IOL injector shown in FIG. 9;

FIG. 15 is a side view of an IOL injector according to a third exemplary embodiment of the disclosure;

FIG. 16 is a side view of the IOL injector shown in FIG. 15 with a partially removed housing;

FIG. 17 is a side view of the IOL injector shown in FIG. 15 with the housing removed;

FIG. 18 is a detailed side view of the timer mechanism of the IOL injector shown in FIG. 15;

FIG. 19 is a bottom view of the timer mechanism of the IOL injector shown in FIG. 15;

FIG. 20 is a cross-sectional side view of the timer mechanism of the IOL injector shown in FIG. 19 illustrating the braking mechanism;

FIG. 21 is a side view of an IOL injector according to a fourth exemplary embodiment of the disclosure;

FIG. 22 is a detailed side view of the timer mechanism of the IOL injector shown in FIG. 21;

FIG. 23 is another detailed side view of the timer mechanism of the IOL injector shown in FIG. 21;

FIG. 24 is a side view of an IOL injector showing a spring compression mechanism with a pusher rod;

FIG. 25 is a cross-sectional bottom view of the IOL injector shown in FIG. 24 with the pusher rod unloaded;

FIG. 26 is another cross-sectional bottom view of the IOL injector shown in FIG. 24 with the pusher rod loaded;

FIG. 27 is a side view of the IOL injector shown in FIG. 26 with a partially removed housing showing a spiral spring; and

FIG. 28 is a side view of the IOL injector shown in FIG. 27 with the housing removed showing a spiral spring.

### DESCRIPTION OF EXEMPLARY EMBODIMENTS

Various objects, features, aspects and advantages of the present disclosure will become more apparent from the following detailed description of the exemplary embodiments of the disclosure, along with the accompanying drawings in which like numerals represent like components.

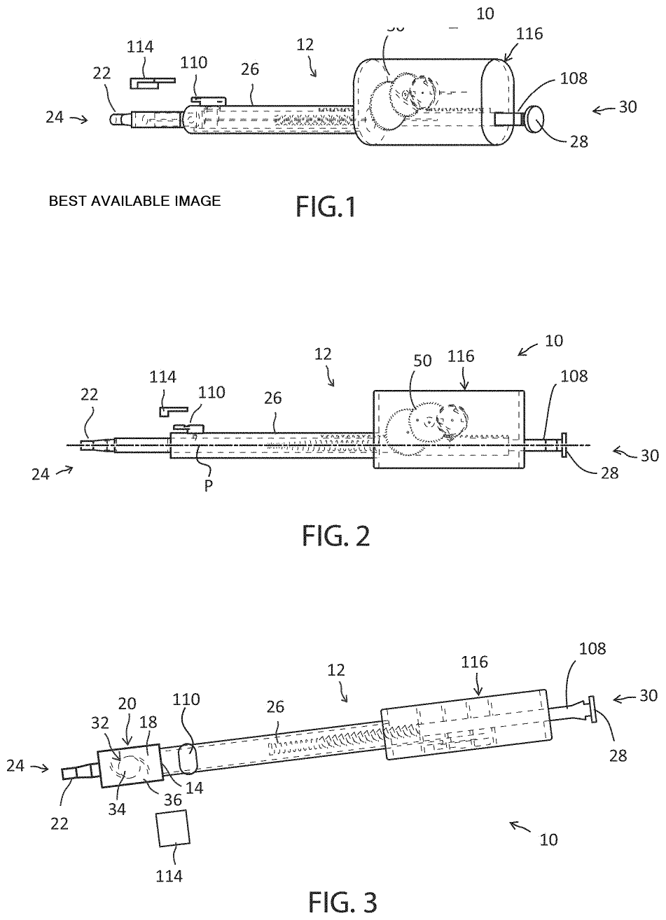

FIGS. 1 to 8 show different views of an IOL injector 10 according to a first exemplary embodiment of the disclosure. The IOL injector 10 has a proximal end 30 and a distal end 24. Injector 10 includes an injector body 12 having a channel 14 extending in a direction of a longitudinal axis P, and a pusher rod 16 slidably inserted in the injector body 12. The injector 10 further includes a cavity 18 for inserting an IOL 32, and a cartridge 20 with an injection nozzle 22 at distal end 24. The injector body 12 has finger grips 26.

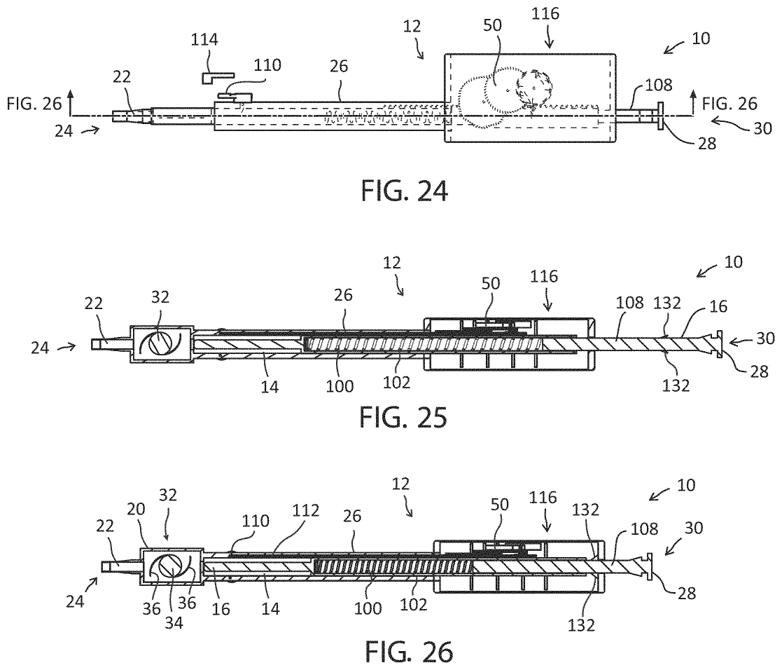

An end of pusher rod 16 opposite from the distal end 24 of the IOL injector 10 mechanically engages with a hollow shaft 102 receiving an axially disposed coiled compression spring 100. Hollow shaft 102 also includes a rack portion 54 of a rack and pinion mechanism 51. Spring 100 can be compressed inside shaft 102 by a spring pusher rod 108 to apply a force onto shaft 102 directed toward the distal end 24. The spring pusher rod 108 can be a fixed part of injector body 12 with spring 100 factory pre-tensed ready for operation. Alternatively, as shown in FIGS. 24 to 26, spring pusher rod 108 can also be an axially slidable part inside the hollow portion of hollow shaft 102 to compress spring 100 when pushed toward the distal end 24 of the IOL injector 10 along longitudinal axis P. A thumb plate 28 on the proximal portion of the spring pusher rod 108 can allow a user to push spring pusher rod 108 to compress spring 100 far enough for operation.

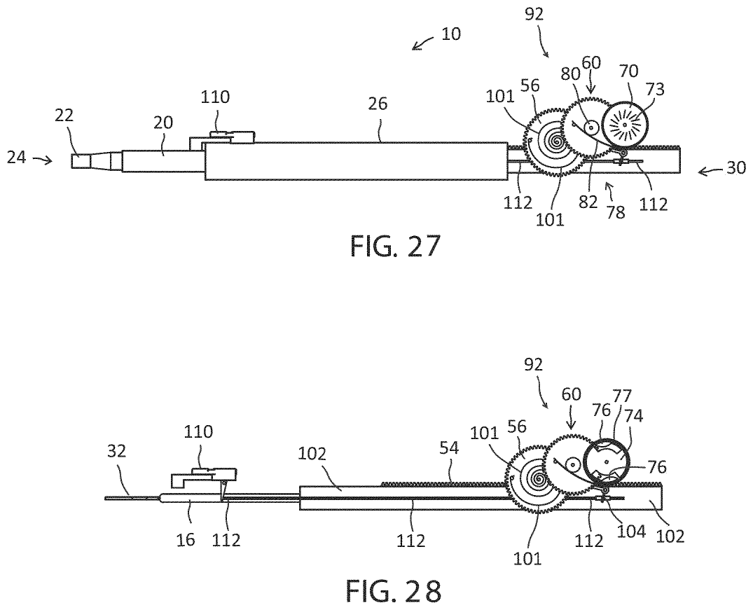

IOL injector 10 further includes pusher rod locking tabs 132 and speed regulator housing 116. The pusher rod locking tabs 132 retain spring pusher rod 108 in a locked position thereby preventing recoil of spring pusher rod 108 after releasing pressure from thumb plate 28 and permitting compressed spring 100 to expand inside the speed regulator housing 116. As an alternative to coil spring 100 directly pushing pusher rod 16, coil spring 100 can be replaced by a pre-tensed spiral spring 101 arranged on one compound gear of the gear train 92, e.g., as shown in FIGS. 27 and 28 to provide mechanical power through pinion 55 and rack 54 to axially displace pusher rod 16 and pushing the IOL toward the eye.

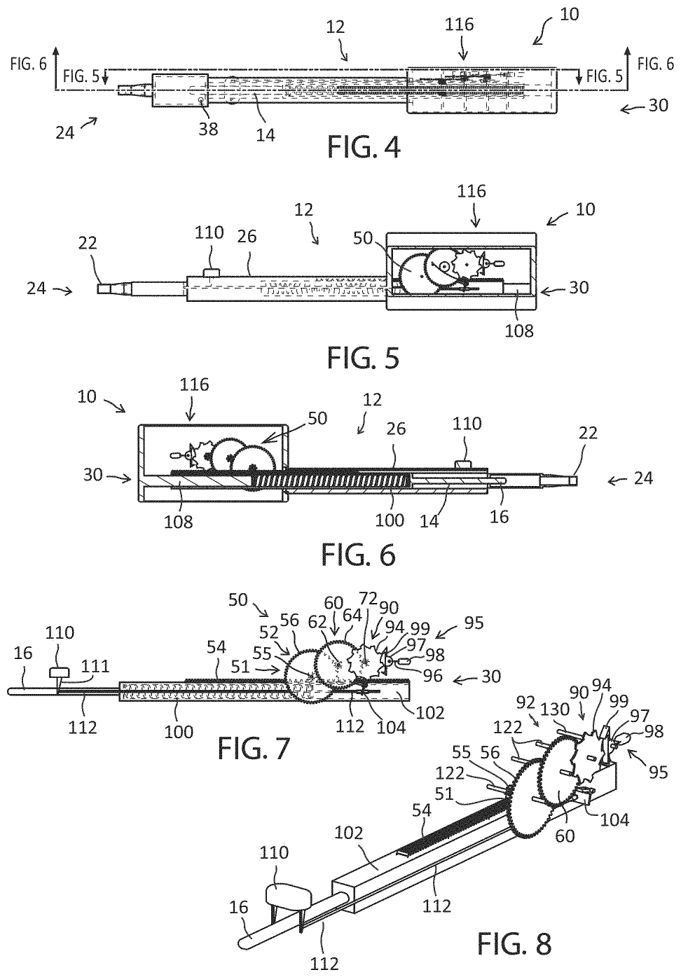

Returning now to FIG. 4. FIG. 4 shows channel 14 extending through the injector body 12, the cavity 18, and the cartridge 20, and which exits at the distal end 24 from injection nozzle 22, such that the pusher rod 16 may advance an IOL 32 positioned in the cavity 18. The term “pusher rod” describes any component advanced through the channel 14 to push an IOL 32 through the cavity 18 and the cartridge 20, such that the intraocular lens 32 exits the IOL injector 10 at the distal end 24 of the injection nozzle 22. In an exemplary embodiment of the disclosure, the injector body 12 may be a reusable hand piece, whereas the cartridge 20, which is releasably connected with the injector body 12, may be a single use part. In another exemplary embodiment, injector body 12, cartridge 20, and cavity 18 may be formed as a single piece from a suitable material, which may include, for example polypropylene or polyethylene. IOL Injector 10 can be made as a fully disposable single use part. Cartridge 20 may also be treated with a lubricious coating, a viscoelastic material and/or liquid in order to facilitate advancement of the IOL within the channel of cartridge 20. Lubricant can be injected in cartridge 20 through a lubricant port 38.

IOL 32 may be any intraocular lens formed of a flexible material, including but not limited to hydrogels, silicone, or acrylic materials. IOL 32 includes as least one optic part 34 and haptics 36 that stably fixate IOL 32 within the eye when implanted. Each haptic 36 has one end anchored in the lens optic part 34 and a free end for attachment to the eye tissue. The optic part 34 is structured to focus light onto a patient's retina and may include any suitable refractive and/or diffractive elements.

FIGS. 3 and 11 show an IOL 32 positioned in cavity 18 and ready to be inserted from cartridge 20 through nozzle 22 pushed by pusher rod 16 into an eye.

As shown in FIGS. 4 to 8, a mechanical speed regulator mechanism 50 including a reduction gearbox is arranged in a speed regulator housing 116 and mechanically coupled with a rack and pinion mechanism 51 to rack 54 of shaft 102 via a first pinion 55 of a first compound gear 52 having a coaxial first large gear 56. Large gear 56 of compound gear 52 mechanically couples with a second pinion 62 of second compound gear 60 also having a second large gear 64. Second large gear 64 mechanically couples with a third pinion 72 of the third compound gear with an escapement wheel 90. The first, second, and third compound gears 52, 60, and 90 form a gear train 92 in a speed multiplying configuration. The escapement wheel 94 of the compound gear with escapement wheel 90 mechanically engages with an anchor 99 emerging from an anchor body 97. First, second, and third compound gears 52, 60, and 90 are each mounted on respective shafts 122 for expedite rotation. Anchor body 97 is mounted on an anchor shaft 130 defining a center of oscillation of anchor 99. Anchor body 97 also receives a proximal end of a pendulum arm 96. Pendulum arm 96 is attached through the distal end to a mass 98 configuring a pendulum 95. Pendulum arm 96 can be configured to be flexible and can be made, e.g., of a steel filament, a plastic sheet, or other suitable flexible material, in a way that the time-constant of the anchor mechanism can be adjusted by selecting a mass 98 weight, a pendulum arm 96 length, and a pendulum arm 96 spring constant. A finger depressible control lever 110 can be arranged on body 12 for a user to be depressed to activate injector 10 for injecting IOL 32 into a capsular bag of an eye. Lever 110 when depressed perpendicular to axis P can push rearward a rod 112 at the distal end by the action of a wedge 111. Control rod 112 can be slidably disposed in a channel parallel to axis P. Control rod 112 runs through a hole 106 in a pivoting gear lock 104 with a gear lock extension 105 pivotable around a gear lock shaft 124.

Control rod 112 has rod stoppers 120 and 121 at each side of hole 106 in a way that axial displacement of rod 112 initiates a pivoting motion on gear lock 104 around shaft 124. Rod 112 is actuated upon by a prestressed compression spring 113 disposed to push rod stopper 121 in the forward direction towards the distal end 24 of the IOL injector 10. With control lever 110 undepressed, gear lock extension 105 remains positioned against the teeth of large gear 64 impeding rotation of gear 64. With control lever 110 in a depressed state, gear lock extension 105 rotates away from the teeth of large gear 64 allowing rotation of gear 64. A safety tab 114 can be inserted between control lever 110 and body 102 in a way such that unwanted depression of lever 110 is prevented.

To operate the IOL injector 10 shown in FIGS. 1 to 8, an operator may hold injector body 12 with one hand to first inject a volume of through lubricant port 38 that is sufficient to fill channel 14 in the region of IOL 32, cavity 18, and nozzle 22 with the viscoelastic substance. Safety tab 114 can be removed with the other hand by the operator to unlock control lever 110 for operation.

Under visual supervision by the operator, lever 110 can be depressed by one finger of the operator with nozzle 22 at the distal end 24 inserted in a surgical incision for delivering the IOL 32 into an eye, typically filled with viscoelastic substance. Depression of lever 110 produces a rearward displacement of control rod 112 running through hole 106 of pivoting gear lock 104 which has extension 105 engaged with a tooth from gear 64 part of second compound gear 60 preventing gear 60 rotation at rest. The rearward displacement of rod 112 pushes the front rod stopper 120 toward the body of gear lock 104 producing a pivoting motion around shaft 124 that has the effect of displacing gear lock extension 105 away from the teeth of gear 64 unlocking compound gear 60 and allowing it to rotate. The rearward displacement of rod 112 also pushes rear rod stopper 121 further compressing prestressed rod spring 113. Release of lever 110 allows spring 113 to force sliding of rod 112 back into the resting position with extension 105 now pivoting towards gear the teeth of gear 64 and back into a locking position of gear 60. In this way an operator can activate and deactivate IOL injection in a controllable manner.

The speed multiplying gear train 92 composed of first, second, and third compound gears 52, 60, and 90 is powered for rotation by the action of compressed spring 100 inside shaft 102 transmitting force to gear train 92 through rack 54 and pinion 55 mechanism 51. When compound gear 60 is in an unlocked state, rotary force is transmitted to compound gear 90. Escapement wheel 94 of gear 90 forces anchor 99 to oscillate. Anchor 99 limits the speed of rotation of gear 90 according to the period of oscillation of anchor 99. The period of oscillation of anchor 99 can be varied by the length of arm 96, the spring constant of arm 96, and the weight of mass 98. Other factors can also vary the period of oscillation of anchor 99. In a typical configuration, the period of oscillation of anchor 99 can be adjusted to 0.1 seconds (s) (which is the same as a frequency of 10 oscillations per second). The gear train can be configured to advance the pusher rod 16 at a speed of 3 millimeters per second (mm/s) with these settings.

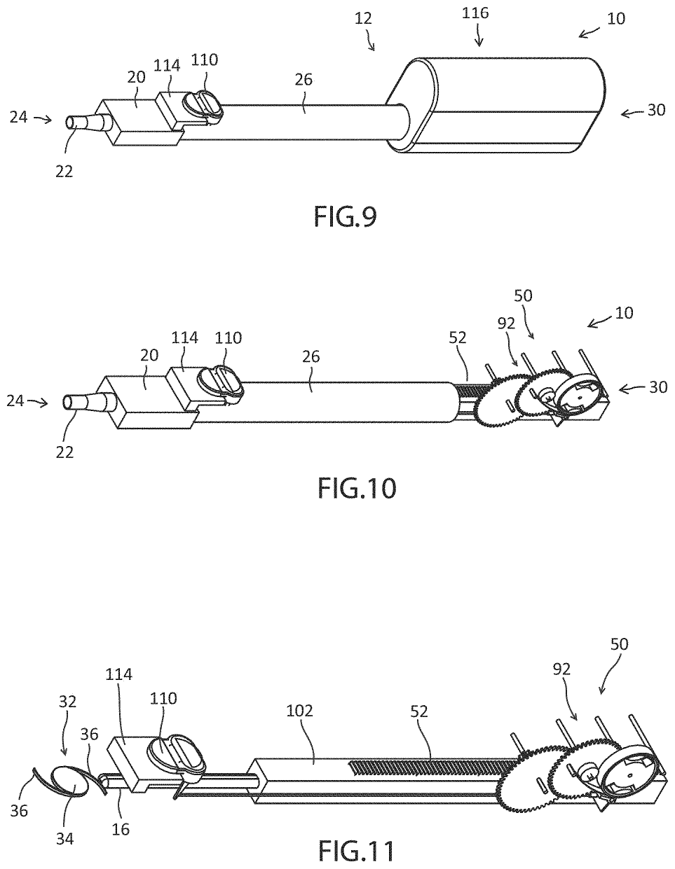

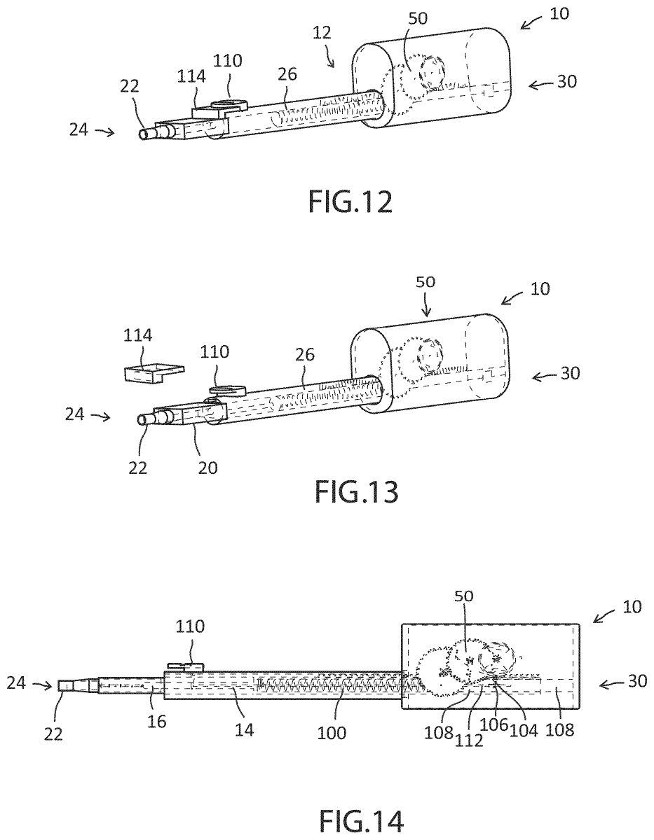

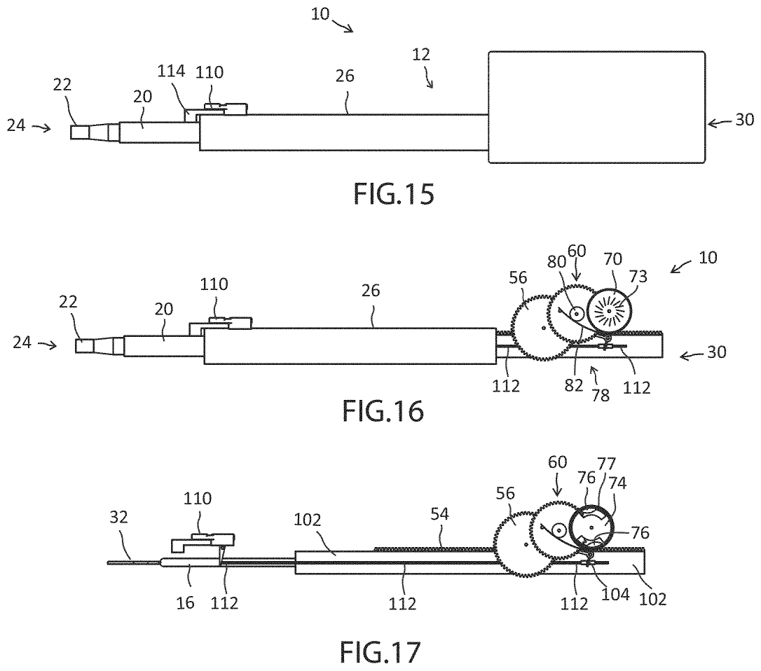

FIGS. 9 to 14 show different views of an IOL injector 10 according to a second exemplary embodiment of the disclosure, and FIGS. 15 to 20 show different views of an IOL injector 10 according to a third exemplary embodiment of the disclosure. In the exemplary embodiments shown in FIGS. 9 to 20, the third compound gear of escapement wheel 90 is replaced by a third compound gear 70 including flywheel 74. More specifically, compound gear 70 includes third pinion 72 and flywheel 74.

Flywheel 74 introduces an inertial component that allows for incremental axial speed of pusher rod 16 once control lever 110 is depressed by an operator. A maximum speed of rotation of flywheel 74 can be achieved that is within a maximum tolerated speed using the simple flywheel 74 embodiment. The operator can release lever 110 to stop the advancement of pusher rod 16 if the speed is determined to be too fast and the action can be repeated to start again with pusher rod 16 moving from the detent state at incremental speed.

Further control can be obtained by incorporating a speed limiting feature into the flywheel 74. One example of improved control of the maximum speed of rotation of the flywheel 74 by incorporating a speed limiting feature 75 is shown in FIG. 16 where flywheel 74 incorporates air resistive fins 73 which limit the maximum speed of rotation of flywheel 74 based on the principle of increasing air friction with speed of rotation which results in an equilibrium speed of flywheel 74 that can be tailored to the desired maximum speed.

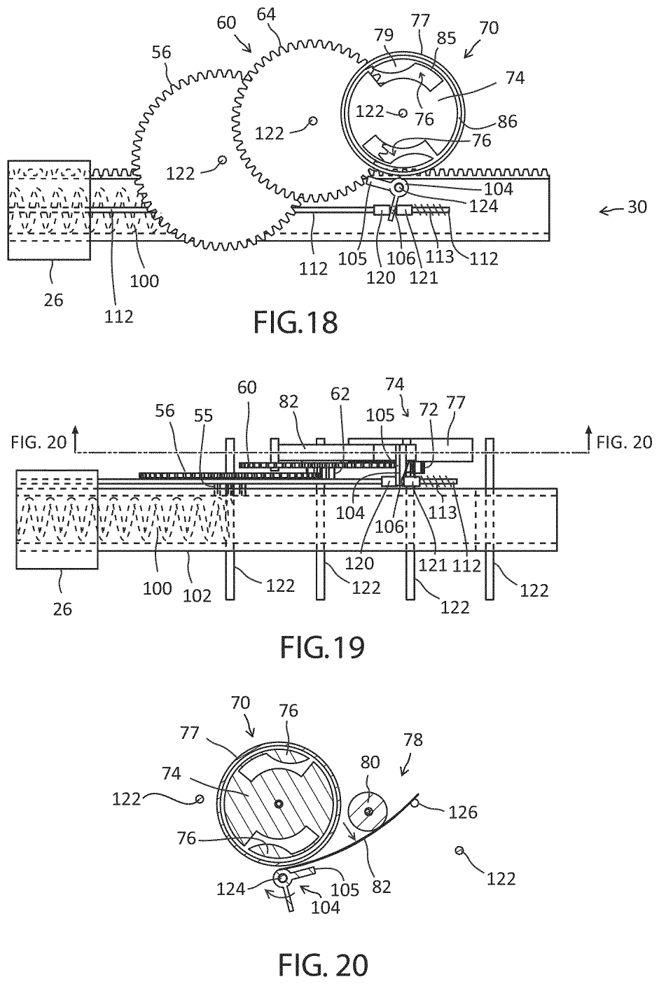

An exemplary embodiment of an improved maximum speed control of flywheel 74 is shown in FIG. 18. In this exemplary embodiment, flywheel 74 includes at least one centrifugal speed limiter 76 providing a speed limiting feature. Each of the at least one centrifugal speed limiters 76 includes a flexible arm 85 mounted on the body of flywheel 74 and a centrifugal mass 79 arranged on an end of the radially flexible arm 85 facing away from the body of the flywheel 74. A flywheel shell 77 encircles flywheel 74 defining a circumferential clearance space 86. Flywheel shell 77 can be an extension of speed regulator housing 116. Flywheel 74 can rotate up to a speed where the centrifugal force displaces centrifugal masses 79 thereby bending the radially flexible arms 85 across the clearance space 86. At this critical speed, the centrifugal masses 79 become in contact with flywheel shell 77 and a frictional force slows down flywheel 74 limiting the maximum speed. A maximum flywheel 74 speed can be preset by adjusting the centrifugal masses 79, the flexibility of arms 85, and the dimensions of clearance space 86.

Another exemplary embodiment of an improved speed control is shown in FIGS. 16, 17, and 20. In this exemplary embodiment, depressing lever 110 first releases gear lock extension 105 from the teeth of gear 64. A friction brake 78 is composed of a friction band 82 in contact with a friction wheel 80 built into compound gear 60. Friction band is fixed on one end through a friction band anchor stud 126. The opposite end of friction band 82 is anchored to gear lock 104 in a way such that incremental rotation of lock 104 produces progressive loosening of friction band 82 against friction wheel 80 with a progressive release of brake 78 and a progressive speed of flywheel 74.

In this way, incrementally depressing lever 110 by an operator can produce proportional loosening of band 82 with proportional speed control of flywheel 74 and of pusher rod 16.

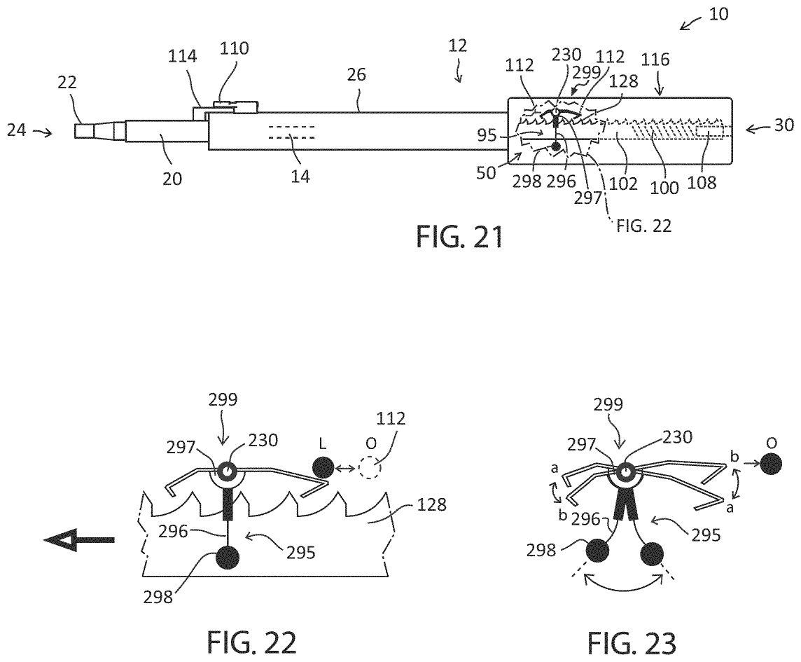

In FIGS. 21 to 23 an IOL injector 10 having a body 12 including a speed regulator mechanism 50 according to a fourth exemplary embodiment of the disclosure. In this exemplary embodiment, escapement wheel 94 has been replaced by an escapement rack 128 arranged alongside hollow shaft 102. Gear train 92 has been removed and anchor body 297 is mounted on an anchor shaft 230 such that anchor 299 is operatively coupled with escapement rack 128 of hollow shaft 102. By depressing the control lever 110 by an operator, control rod 112 slides rearward from a locked position L blocking oscillation of anchor 299 to an operate position 0 at which anchor body 297 is released to freely oscillate around the anchor shaft 230 from position “a” to “b” and reverse, see FIG. 23, powered by the force of spring 100 compressed by spring pusher rod 108 and transmitted through hollow shaft 102 to escapement rack 128. Pendulum 295 has a pendulum arm 296 configured to act as a spring, and a pendulum mass 298. The arm length of the pendulum arm 296, the arm spring constant, and the mass value of the mass 298 are adjusted such that the pendulum 295 swings at a rate at which the shaft 110 and the pusher rod 16 advance at a maximum speed, typically at a speed of millimeters per second 3 mm/s.

The injector body 12 holds the cartridge 20. The cartridge 20 can be releasably attached to the injector body 12. The cartridge 20 can be delivered separately. The injector body 12 including pusher rod 16 can be a multiuse device, whereas the cartridge 20 can be a single use device. The injector body 12 and the cartridge 20 can also be manufactured as one single part.

It is understood that the foregoing description is that of the exemplary embodiments of the disclosure and that various changes and modifications may be made thereto without departing from the spirit and scope of the disclosure as defined in the appended claims.

### LIST OF REFERENCE NUMERALS

P longitudinal axis
10 IOL injector
12 body
14 channel
16 pusher rod
18 cavity
20 cartridge
22 nozzle
24 distal end
26 graspable portion
28 thumb plate
30 proximal end
32 intraocular lens
34 optic part
36 haptics
38 lubricant port
50 speed regulator mechanism
51 rack and pinion mechanism
52 first compound gear
54 rack
55 first pinion
56 first large gear
60 second compound gear
62 second pinion
64 second large gear
70 third compound gear with flywheel
72 third pinion
73 air resistive fins
74 flywheel
75 speed limiting feature
76 centrifugal speed limiters
77 flywheel shell
78 friction brake
79 centrifugal mass
80 friction wheel
82 friction band
85 radially flexible arm
86 clearance
90 third compound gear with escapement wheel
92 gear train
94 escapement wheel
95 pendulum
96 pendulum arm
97 anchor body
98 mass
99 anchor
100 spring
101 spiral spring
102 hollow shaft
104 pivoting gear lock
105 gear lock extension
106 hole
108 spring pusher rod
110 control lever
111 wedge
112 control rod
113 rod spring
114 safety tab
116 speed regulator housing
120 front rod stopper
121 rear rod stopper
122 gear shaft
124 gear lock shaft
126 friction band anchor stud
128 escapement rack
130 anchor shaft
132 pusher rod locking tabs
230 anchor shaft
295 pendulum
296 anchor arm
297 anchor body
298 anchor mass
299 anchor

What is claimed is:

1. An intraocular lens (IOL) injector for releasing an IOL to be injected into a capsular bag of an eye, the IOL injector having a proximal end and a distal end, the IOL injector comprising:
an injector body defining a longitudinal axis and including a hollow shaft;
a spring arranged in the hollow shaft;
a cartridge arranged on the injector body and defining a cavity for holding the IOL;
an injection nozzle arranged on the cartridge at the distal end of the IOL injector;
a channel extending through the injector body, the cavity, the cartridge, and the injection nozzle in an axial direction along the longitudinal axis;
a mechanical speed regulator system configured to control a speed of the releasing of the IOL;
a pusher rod engaged via the hollow shaft with the mechanical speed regulator system and arranged in the channel movably in the axial direction by a force generated by the spring to advance the IOL in the axial direction towards the distal end of the IOL injector out of the cavity through the cartridge and the injection nozzle;
a control lever arranged on the injector body in communication with the mechanical speed regulator system;
the mechanical speed regulator system being responsive to an actuation of the control lever;
the mechanical speed regulator system includes a first compound gear, a second compound gear, and a third compound gear,
the first compound gear includes a first pinion and the hollow shaft includes a rack;
the first pinion and the rack form a rack and pinion mechanism to mechanically couple the mechanical speed regulator system with the hollow shaft,
the first, second, and third compound gears form a gear train, and
the gear train has a speed multiplying configuration;
wherein:
the mechanical speed regulator system further includes an anchor mechanism with an anchor, and
the third compound gear includes an escapement wheel mechanically engaging with the anchor.

2. The IOL injector of claim 1, wherein:
the anchor mechanism includes an anchor shaft defining a center of oscillation of the anchor,
the anchor has an anchor body, and
the anchor body is mounted on the anchor shaft and swivels about the anchor shaft.

3. The IOL injector of claim 2, wherein:
the anchor mechanism further includes a pendulum arm arranged on the anchor body and a mass arranged on an end of the pendulum arm facing away from the anchor body.
